(19) United States
(12) Patent Application Publication
(10) Pub. No.: US 2008/0243156 A1
(43) Pub. Date: Oct. 2, 2008

(54) Ophthalmic surgical instrument & surgical methods

(76) Inventor: Thomas John, Oak Lawn, IL (US)

Correspondence Address: CONNORS ASSOCIATES, 1600 DOVE ST, SUITE 220, NEWPORT BEACH, CA, 92660, US

(21) Appl. No.: 11/731,811

(22) Filed: Mar. 30, 2007

(51) Int. Cl. A61F 9/007

(52) U.S. Cl. 606/166

(57) ABSTRACT

Surgical instruments and their methods of use enable a surgeon to form a precut lamellar disk that is removed by the same through a single incision along the perimeter of the cornea. This may be done using one this one instrument. Another instrument enables the surgeon to scrap off adhering parts. One more instrument enables the surgeon to hold a folded cornea donor disk to avoid inverting the disk upon unfolding. Yet another instrument enables the surgeon to “iron out” wrinkles in the implanted donor cornea.

### INCORPORATION BY REFERENCE

Any and all U. S. patents, U. S. patent applications, and other documents, hard copy or electronic, cited or referred to in this application are incorporated herein by reference and made a part of this application.

### DEFINITIONS

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

The word “rectangular ” includes square.

### BACKGROUND

One way to treat a cornea clouded by an injured or dysfunctional endothelium was a full-thickness corneal transplant. This standard penetrating keratoplasty procedure worked well enough. But there were serious drawbacks—among them slow healing, major induced astigmatism, risk of ulcer, erosion, suture-related infections, and a permanently weakened cornea. Indeed, it was not uncommon for corneal surgeons to see patients rupture a transplant wound or even lose an eye in a fall or other minor trauma, often years after the surgery.

In the late 1990s, a Dutch surgeon, Gerrit Melles, MD, pioneered a procedure in which the inner most layers (stroma, endothelium, and Descemet's membrane) of the cornea were manually dissected into a lamellar disk comprising a series of layers with the endothelium sandwiched between the stroma and the Descemet's membrane. The lamellar disk is removed to create a circular, aspheric, posterior recess in the stroma of the cornea. A donor corneal disk having a diameter dimension substantially the same as the removed lamellar disk is inserted into the recess. The procedure, known as posterior lamellar keratoplasty, or PLK, promised quicker recovery, little induced astigmatism, less risk of infection and a cornea much less prone to rupture. PLK requires an extremely precise dissection of a “manhole” recess in the inside surface of the patient's cornea matched by a “cover” donor harvested with equal precision from a donor required an extremely precise dissection of a “manhole” in the inside surface of the patient's cornea matched by a “cover” harvested with equal precision from the donor. Since there is no surface corneal wound and no sutures on the cornea, this corneal transplantation leaves the outer layers of the cornea intact. A superbly delicate touch is required to split the cornea, create a recess within the cornea without perforating the cornea. The original technique and its early successors proved exceedingly difficult to master. Surgeons often had to convert to full-penetration procedures during surgery multiple times on the way up the learning curve. I discuss PLK is my recently published surgical textbook, entitled, “Surgical Techniques in Anterior and Posterior Lamellar Corneal Surgery.”

Nevertheless, the potential advantages of PLK are too good to pass up. Developing an easily reproducible minimally invasive procedure that improves patient outcomes and experience will do as much for corneal transplant surgery as phacoemulsification has done for cataract surgery. Dr. Melles describes a technique for removal of Descemet's membrane and the compromised endothelium instead of a lamellar dissection that simplified the technique. This has been called by various names, namely, DXEK (Descemetorhexis with endokeratoplasty), DSEK (Descemet's membrane stripping endothelial keratoplasty) and DSAEK (Descemet's membrane stripping automated endothelial keratoplasty).

A major reason why PLK is so difficult—even awkward—is a lack of suitable instruments. While PLK is performed on the curved “ceiling” of the eye's anterior chamber, currently available instruments are designed for cataract, glaucoma or retinal surgery—procedures performed on the “floor” of the eye from the surgeon's point of view. Consequently, a jerking motion in using the instruments and multiple incisions for entry into the anterior chamber required in using these “floor” instruments is not only excessively time-consuming and fatiguing, it also interrupts the flow of the procedure, sacrificing the natural control that is best be achieved through a continuous, fluid motion. As I recognize, it is best to remove the endothelium and Descemet's membrane as a single disk every single time if possible, producing the least amount of trauma to the corneal stroma. This, however, is not possible with the existing substantially linear surgical instruments. If the surgeon stops and starts, it is really difficult to pick up the tear. Once the surgeon starts digging, he or she gets strips of stroma hanging down.

### SUMMARY

My instruments and methods have one or more of the features depicted in the illustrative embodiments discussed in the section entitled “DETAILED DESCRIPTION OF SOME ILLUSTRATIVE EMBODIMENTS.” The claims that follow define my invention. Briefly, however, my instruments use a curved arm that facilitates working on the posterior of the cornea. This arm has different types of tip portions depending on the application. One tip portion enables the surgeon to access substantially the entire posterior of the cornea through a single incision and create a precut lamellar disk. This tip portion also enables the surgeon peel off the precut lamellar disk. Other tip potion provide for scrapping, fixation, and wrinkle removal.

### DESCRIPTION OF THE DRAWING

Some embodiments of my surgical instruments and methods are now discussed in detail. These embodiments depict my novel and non-obvious instruments and methods as shown in the accompanying drawing, which is for illustrative purposes only. This drawing includes the following figures (Figs.), with like numerals indicating like parts:

FIG. 1 is a cross-sectional view of a human eye.

FIG. 2 is a series of photographs looking into an eye and showing one embodiment of my surgical “spatula” instrument being used to make a 360-degree cut in the posterior of the cornea and create a single unitary lamellar disk.

FIG. 3 is photograph looking into an eye and showing a wadded up mass of cellular material to be removed.

FIG. 4 is a series of photographs showing a donor corneal disk being folded prior to being inserted into the anterior chamber of the eye depicted in FIG. 2.

FIG. 5 is a series of photographs looking into an eye and showing the folded donor corneal disk shown in FIG. 4 being inserted into the anterior chamber of the eye depicted in FIG. 2.

FIG. 6 is a perspective view of one embodiment of my “spatula” instrument.

FIG. 7 is an enlarged, fragmentary perspective view taken along the line 7 in FIG. 6.

FIG. 8 is a side elevational view of the embodiment of my surgical “spatula” instrument shown in FIG. 6.

FIG. 9 is a cross-sectional view taken along line 9-9 of FIG. 8.

FIG. 10 is a perspective view of one embodiment of my “stripper” instrument.

FIG. 11 is a fragmentary side elevational view of the tip portion of my “stripper” instrument shown in FIG. 10.

FIG. 12 is a top plan view of the tip portion shown in FIG. 11.

FIG. 13 is a perspective view of the tip portion shown in FIG. 11.

FIG. 14 is a side elevational view of the embodiment of my surgical “stripper” instrument shown in FIG. 10.

FIG. 15 is an enlarged, fragmentary perspective view taken along the line 15 in FIG. 14.

FIG. 16 is a perspective view of one embodiment of my surgical “glider” instrument.

FIG. 17 is an enlarged, fragmentary perspective view taken along the line 17 in FIG. 16.

FIG. 18 is an enlarged, fragmentary perspective view of the tip portion shown in FIG. 16.

FIG. 19 is a side elevational view of the embodiment of my surgical “glider” instrument shown in FIG. 16.

FIG. 20 is an enlarged, fragmentary side elevational view of the tip portion of the “glider” instrument shown in FIG. 16.

FIG. 21 is a top plan view of the tip portion shown in FIG. 20.

FIG. 22 is a perspective view of one embodiment of my “fixation hook” instrument.

FIG. 23 is an enlarged, fragmentary perspective view taken along the line 23 in FIG. 22.

FIG. 24 is a side elevational view of the embodiment of my “fixation hook” instrument shown in FIG. 22.

FIG. 25 is an enlarged, fragmentary cross-sectional view of the tip portion of the “fixation hook” instrument shown in FIG. 22.

FIG. 26 is a schematic diagram looking into the eye and depicting the area that can be accessed by a linear surgical instrument through a single incision.

FIG. 26A is a schematic diagram looking into the eye and depicting using my “spatula” instrument to make a cut in the posterior of the cornea along a 360 degree score line.

FIG. 26A is a cross-sectional view of an eye with my “spatula” instrument inserted into the anterior chamber through an incision along the perimeter of the cornea.

FIG. 26B is a cross-sectional view similar to FIG. 26A showing my “spatula” instrument within the anterior chamber and moved to a different position.

FIG. 27 is a cross-sectional view of an eye with my “stripper” instrument inserted into the anterior chamber through an incision along the perimeter of the cornea.

FIG. 28 is a schematic diagram looking into the eye and depicting using my “fixation hook” instrument to hold a folded donor corneal disk as air is introduced to unfolded the donor corneal disk.

FIG. 29 is a cross-sectional view of an eye with my “glider” instrument in one position bearing against the anterior of the cornea.

FIG. 30 is a cross-sectional view similar to that of FIG. 29 showing my “glider” instrument moved to another position and bearing against the anterior of the cornea.

### DETAILED DESCRIPTION OF SOME ILLUSTRATIVE EMBODIMENTS

### General

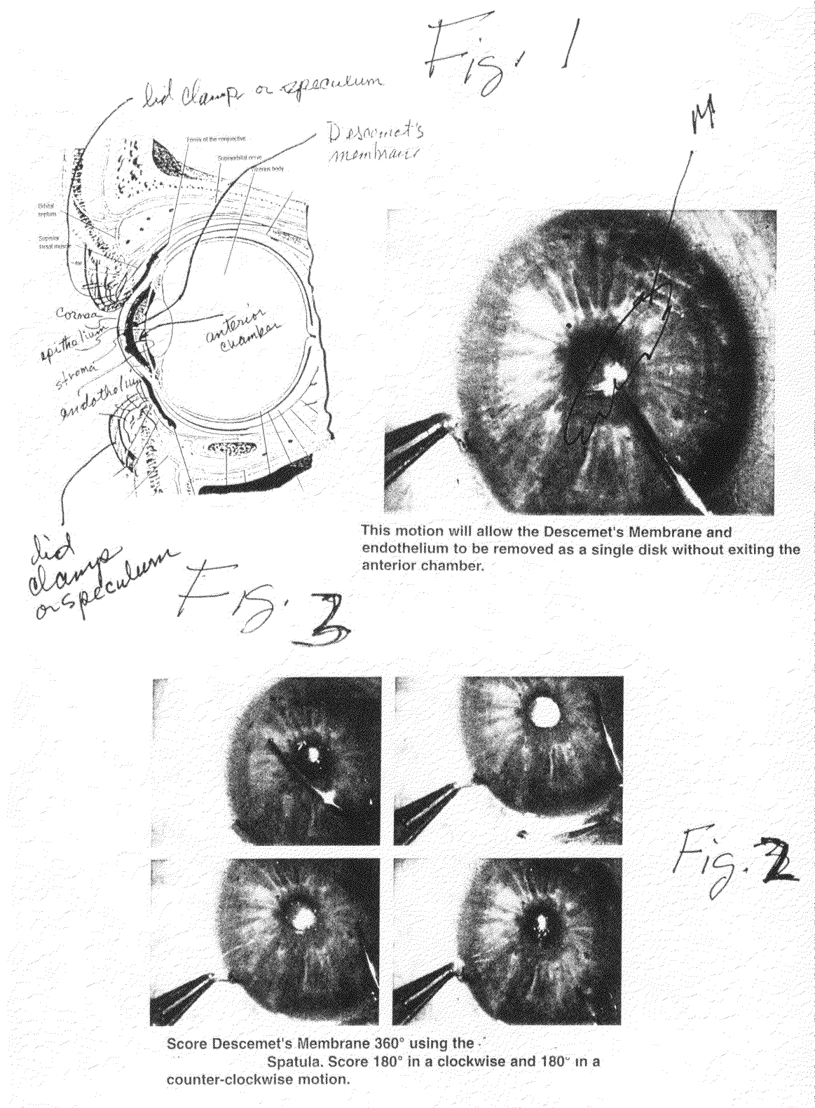

FIG. 1 depicts an eye where lid clamps or speculum pull back and hold the eyelids wide open so a surgeon can operate on the eye's cornea. My ophthalmic surgical instruments enable an eye surgeon to conduct PLK surgery using my improved ophthalmic surgical methods.

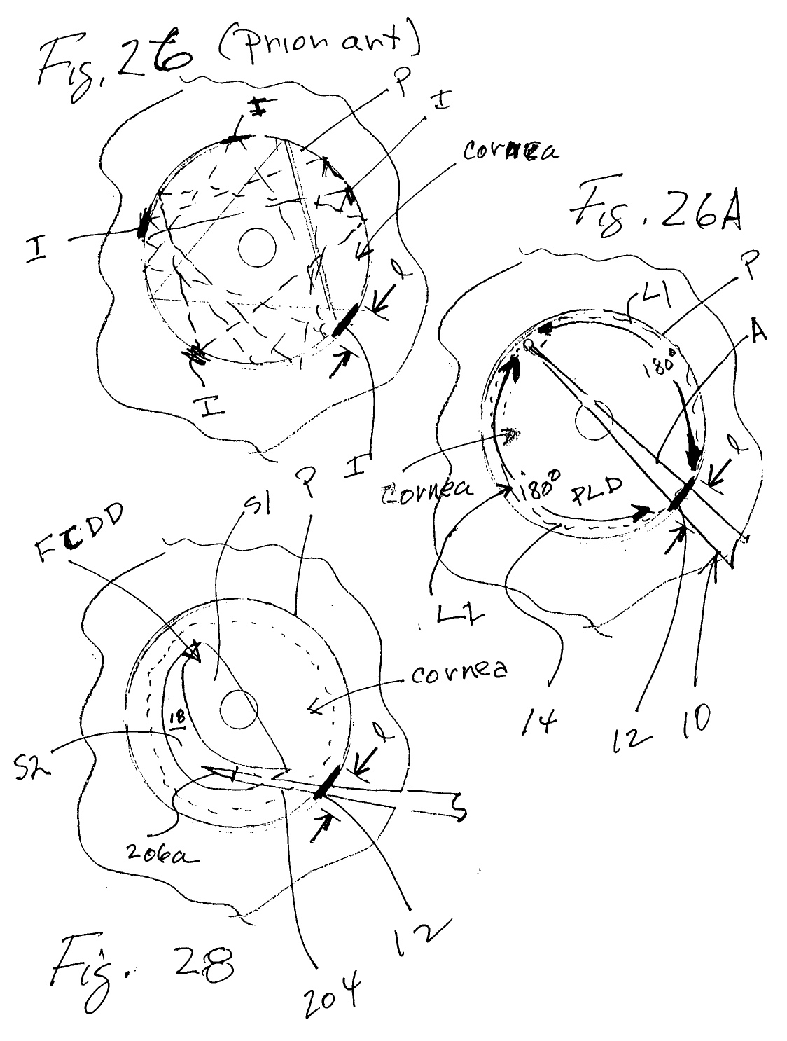

One method employs one embodiment of my “spatula” instrument shown in FIGS. 6 through 9 and generally identified by the numeral 10. Another of my methods employs one embodiment of my “stripper” instrument shown in FIGS. 10 through 15 and generally identified by the numeral 100. One more of my methods employs one embodiment of my “fixation hook” instrument shown in FIGS. 22 through 25 and generally identified by the numeral 200. Still another of my methods employs one embodiment of my “glider” instrument shown in FIGS. 16 through 21 and generally identified by the numeral 300.

As discussed subsequently in greater detail, the “spatula” instrument 10 is used to access through a single incision 12 (FIG. 26A) along the perimeter P of the cornea essentially the entire posterior surface of the cornea. Prior art surgical procedures require more than a single incision 12 along the cornea's perimeter 14, typically 2-3. Such multiple incisions I as shown FIG. 26 enable a linear surgical instrument to be inserted at several different points along the perimeter 14. The areas of the posterior of cornea that such a linear surgical instrument may access from any one incision I are inscribed by a triangle shown in dotted lines. Thus overlapping multiple triangles illustrate that most, if not all, of the posterior of cornea can only be accessed by repeated linear instrument insertion through individual multiple incisions I.

In PLK surgery, even when using my spatula instrument 10, sometimes an internal part of the precut lamellar disk PLD (FIG. 26A) adheres and cannot simply be detached from the cornea's stroma by peeling it away. The surgeon employs the “stripper” instrument 100 to overcome this problem. He or she withdraws the “spatula” instrument 10 from the incision 12 and then inserts the “stripper” instrument 100 through this same incision 12 and uses this instrument to scrap against the adhering internal part of the lamellar disk to detach it. Once this part is detached, the “stripper” instrument 100 is withdrawn from the incision 12 and the “spatula” instrument 10 then used again to continue to peel away the precut lamellar disk PLD.

In PLK surgery the folded cornea donor disk FCDD (FIG. 28) within the anterior chamber of the eye is unfolded by injecting air between overlapping segments S1 and S2 of the folded cornea donor disk. FIG. 4 shows using a forceps to fold a precisely dimensioned circular cornea donor disk. As shown in the FIG. 28, the folded cornea donor disk FCDD is inserted into the anterior chamber through the incision 12. It has been folded so the overlapping segment S1 covers only a part of an underlying segment S2 to expose a surface 18 of this underlying segment. Segment S1 may comprise approximately 40% of the folded cornea donor disk FCDD and segment S1 may comprise approximately 60% of the folded cornea donor disk. With a tip portion 204 of the “fixation hook” instrument 200 on the exposed endothelium surface 18, a wire element 206 is moved into an extended position to press its terminal end 206a against the surface 18, holding this surface so the folded cornea donor disk FCDD does not invert or flip over when air is injected.

In PLK surgery there may appear wrinkles in the cornea after implantation of a cornea donor disk. The “glider” instrument 300 is employed to overcome this problem.

### Spatula Instrument and Method of Use

As best depicted in FIGS. 6 through 9, the spatula instrument 10 includes an elongated handle H1 and a curved tip portion 16 at a distal end of the handle. The tip portion 16 has a predetermined configuration enabling the tip portion to be inserted into the anterior chamber through the incision 12 along the perimeter P of the eye's cornea. In general, the incision 12 has a length of no greater than 5 microns, and ranges substantially from 4.8 to 5 microns. The tip portion 16 has a distal free end 16a that is pointed so, when within the anterior chamber, a surgeon can make a 360 degree substantially circular cut through the cornea's endothelium and Descemet's membrane to form the precut lamellar disk PLD.

The tip portion 16 comprises a rigid arm A, which may comprises compound curves. This rigid arm A may have the following characteristics:

(a) it tapers inwardly from a base end E1 attached to a distal end E2 of the handle H1 to the free end 16a,

(b) it has a length l₁ measured along a straight line between the base end E1 and free end 16a of substantially from 14 to 15 millimeters,

(c) it has an average radius of curvature ARC of substantially from 20 to 25 millimeters,

(d) at or near the base end E1, its has a diameter of substantially from 3 to 4 millimeters, and

(e) at or near the free end 16a, it has a diameter of substantially from 0.75 to 1.2 millimeters.

The free end 16a may point inward toward the handle H1 at an angle of substantially from 35 to 60 degrees with respect to a central longitudinal reference line CL of the instrument 10. The free end 16a may be displaced with respect to the central longitudinal reference line CL. For example, it may be displace a distance d₁ substantially from 5 to 10 millimeters with respect to the central longitudinal reference line CL. The elongated handle H1 and tip portion 16 may have an overall length l₂ substantially from 100 to 120 millimeters. The arm may have an average diameter substantially from 10 to 15 millimeters.

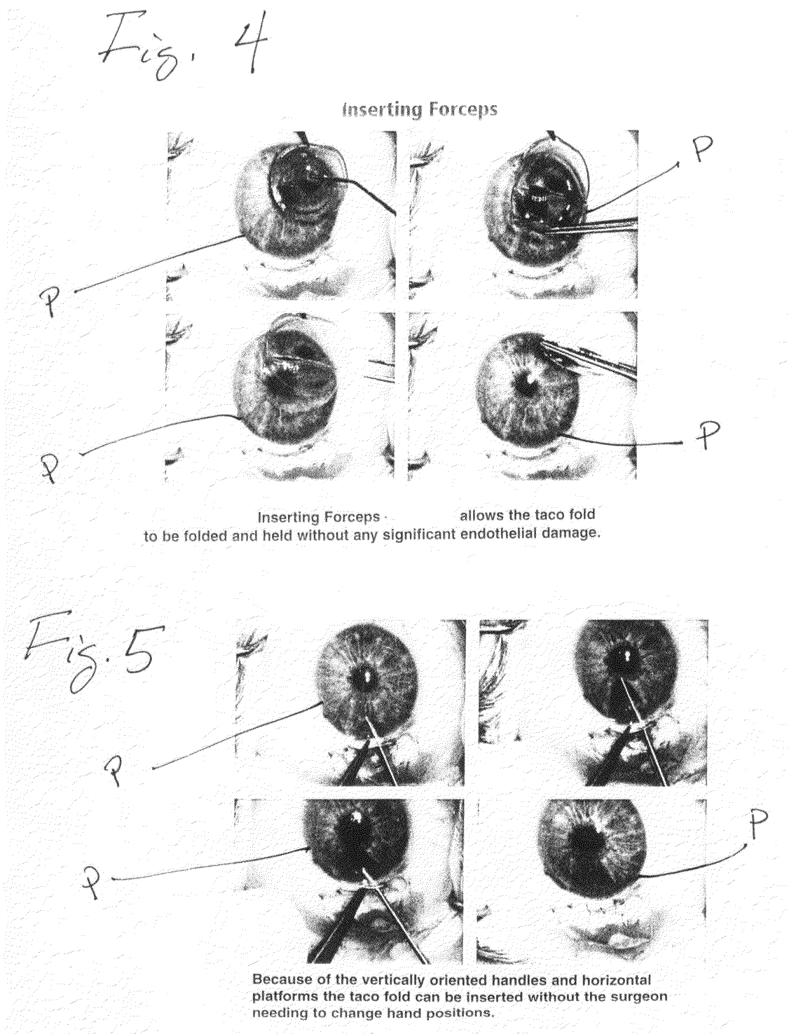

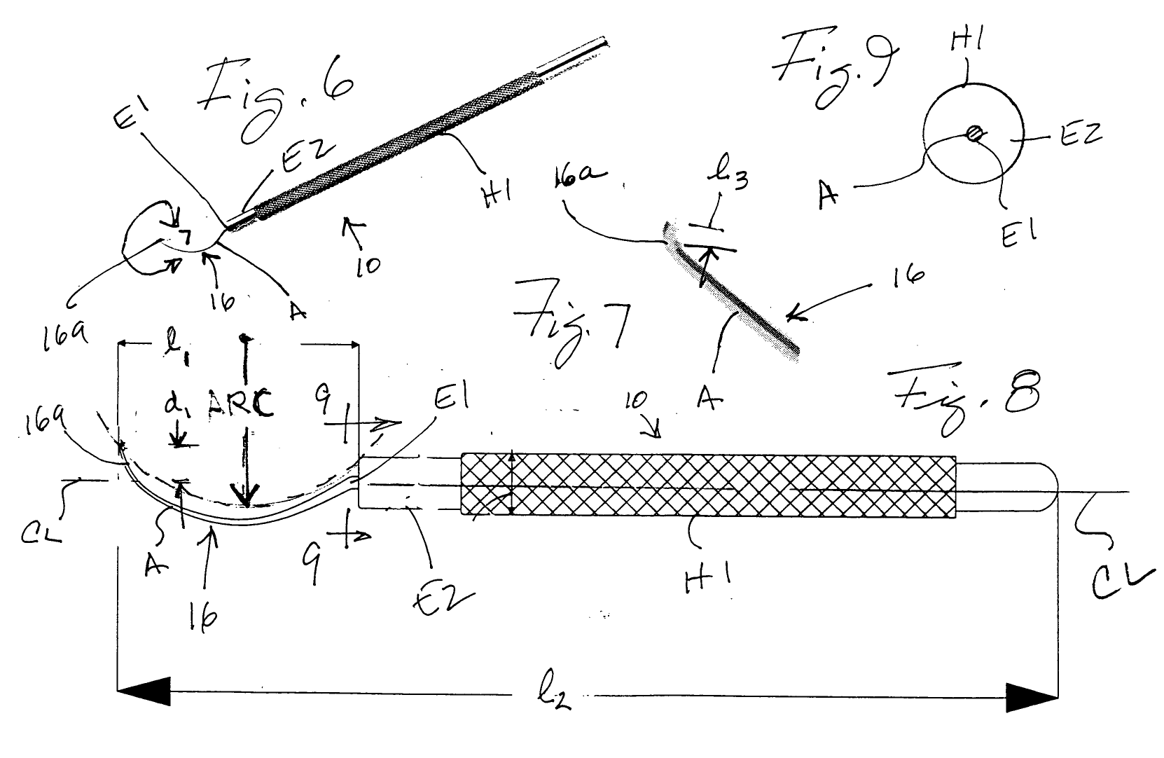

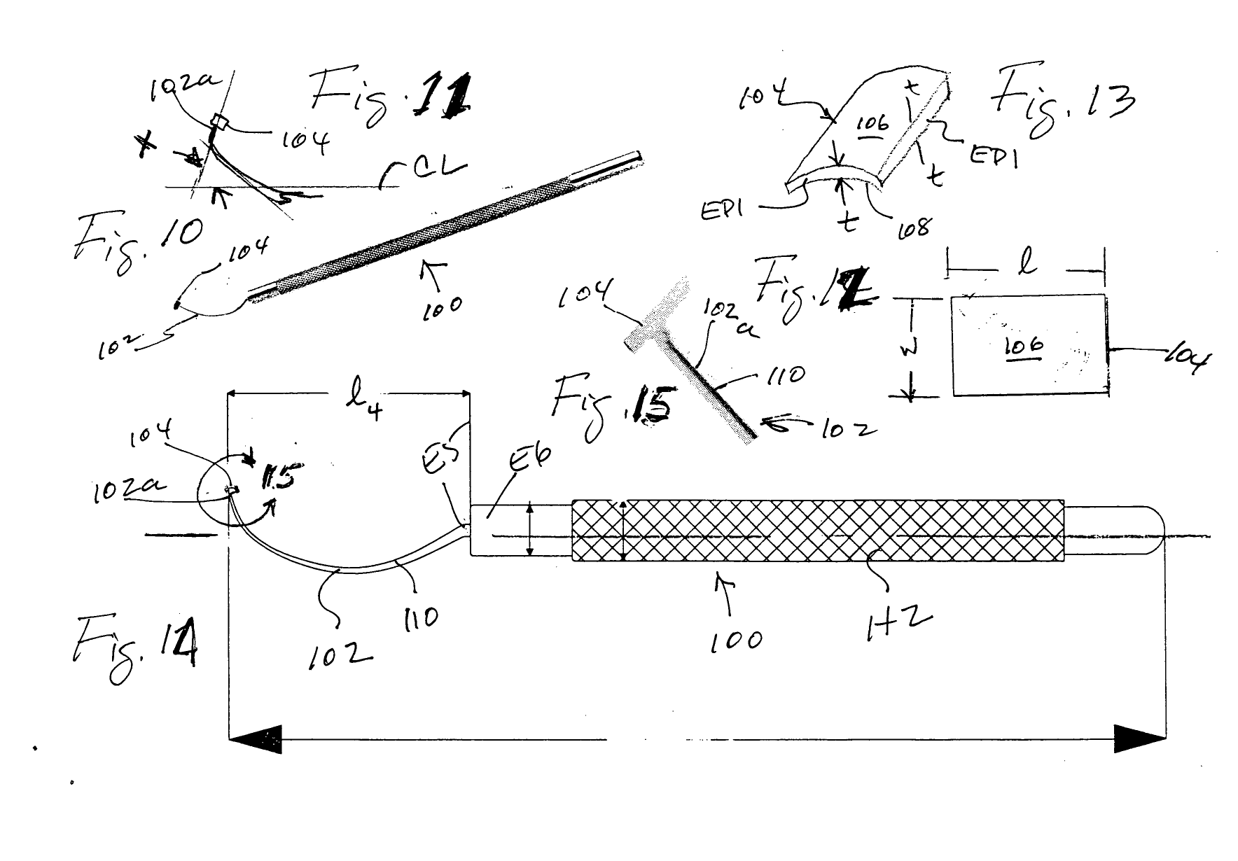

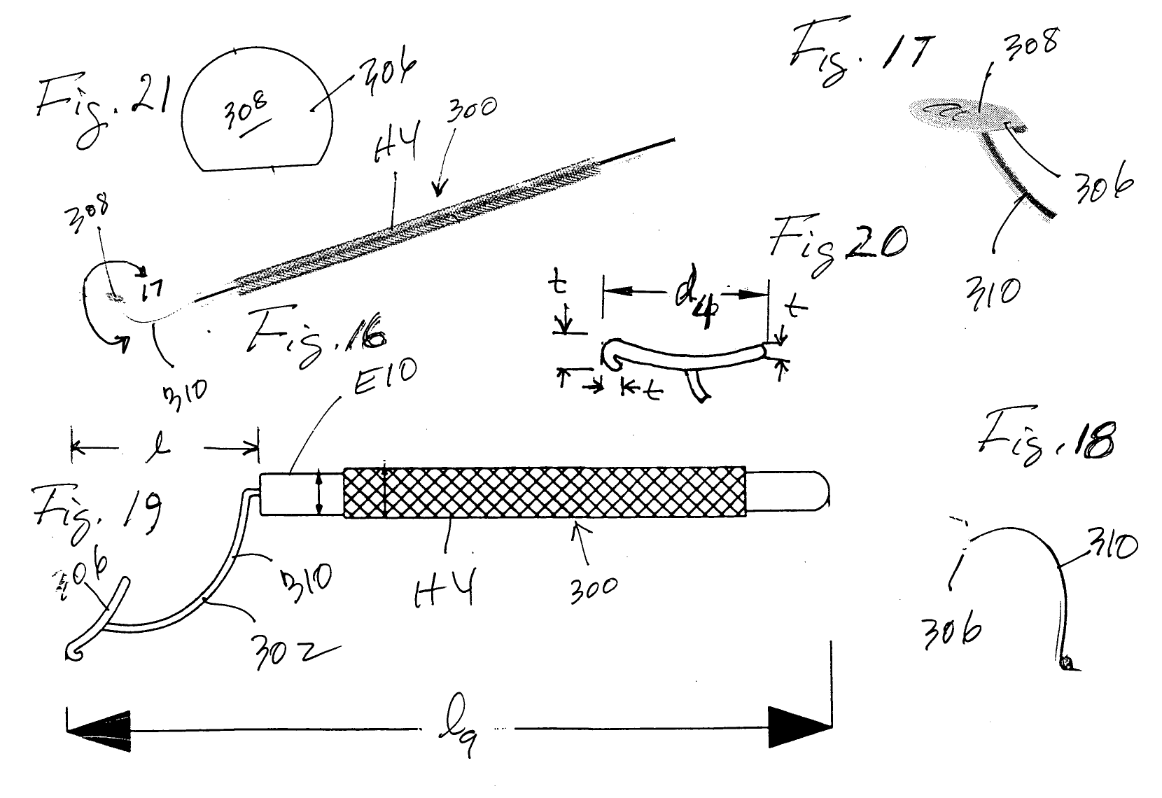

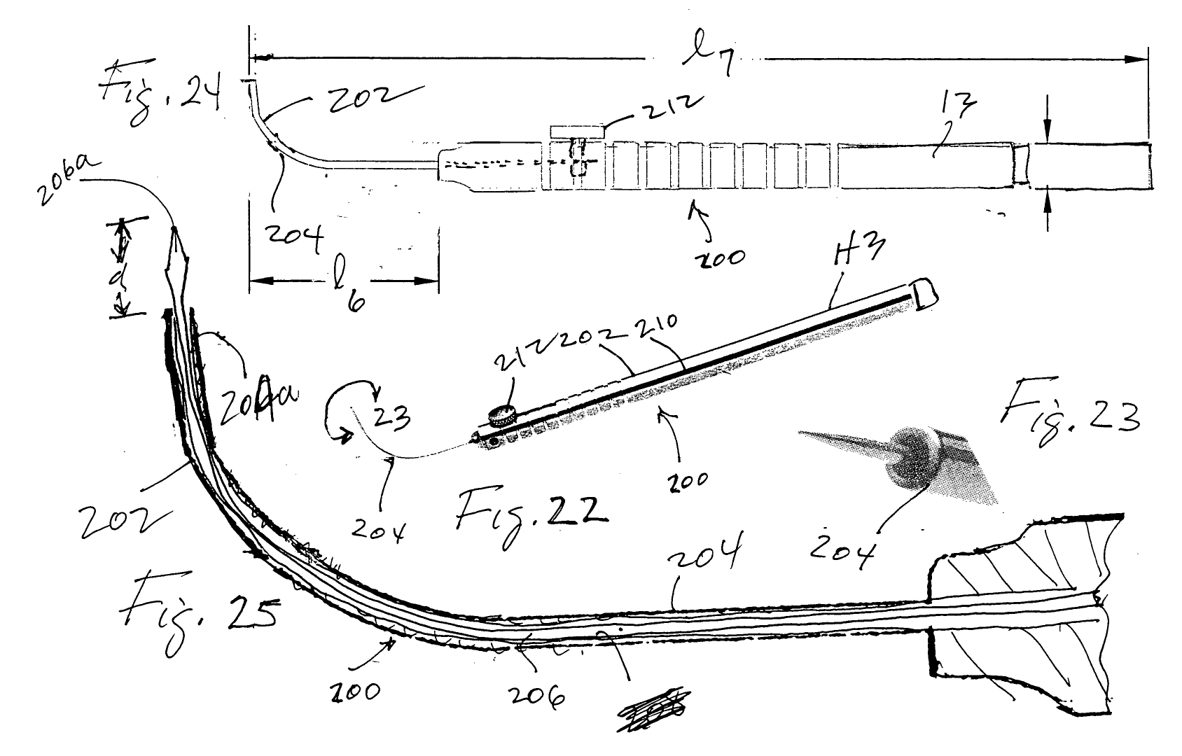

As illustrated in FIGS. 2 and 3 and 26A through 26C, the same spatula instrument 10 is used to create the precut lamellar disk PLD and remove this disk from the anterior chamber. The surgeon makes the 360-degree circular cut in two steps after inserting the tip portion 16 through the incision 12. Step one is to follow a semicircular score line L1 shown in dotted lines in FIG. 26A along the perimeter P in a clockwise direction, pressing the pointed free end 16a against the posterior of the cornea to cut through the cornea's endothelium and Descemet's membrane. Step two is to follow a semicircular score line L2 shown in dotted lines in FIG. 26A along the perimeter P in a counter-clockwise direction, again pressing the pointed free end 16a against the posterior of the cornea to cut through the cornea's endothelium and Descemet's membrane. These steps may be reversed. The length I₃ of the free end 16a is approximately 0.8 1.5 millimeters so it just slightly penetrates into the cornea's stroma in contact with Descemet's membrane as the cut is made. Because of its dimensions the free end 16a cuts into posterior, aspheric underside of the cornea at a depth substantially from 10 to 20 microns.

After forming the precut lamellar disk PLD, the same instrument 10 is used to grasp an edge of the disk and peel the disk away from the underlying stroma of the cornea to expose the surface of the stroma. As the precut lamellar disk PLD is peeled away as depicted in FIGS. 26A and 26B, it is wadded up into a compact mass M of waste cellular material using the spatula instrument 10. As shown in FIGS. 26B and 26C, the surgeon moves the tip portion 16 towards and way from the perimeter P to gather the precut lamellar disk PLD into the compact mass M of waste cellular material. After withdrawing the instrument 10 from the anterior chamber, the mass M is withdrawn through the incision 12 using a forceps inserted through the incision 12. The surgeon grasps the mass M with the forceps and withdraws it through the incision 12 from the anterior chamber.

With the precut lamellar disk PLD completely removed, a central recess CR (FIG. 26C) is formed in the posterior of the cornea exposing a central piece of the stroma that was in direct in contact with the Descemet's membrane prior to removal of the precut lamellar disk PLD. As discussed subsequently in greater detail, the cornea donor disk to be implant is positioned adjacent this recess CR. A conventional stromal scrubber instrument may be used to treat the surface of the exposed stroma prior to implantation.

### Stripping Instrument and Method

As depicted in FIGS. 10 through 15, the stripper instrument 100 is designed to detach an adhering internal part AP of a precut lamellar disk PLD that cannot simply be detached from the cornea's stroma by peeling away the disk from the stroma. This instrument 100 includes a handle H2 and a curved tip portion 102 having a predetermined configuration enabling the tip portion to be inserted into an anterior chamber of an eye through the incision 12. The tip portion 102 terminates in a distal free end 102a having a substantially T-shaped configuration including a plate member 104 having thin cutting edges ED1 for scrapping against the adhering internal part AP to detach this part from the stroma. The plate member 104 has an overall substantially rectangular shape including an outer convex surface 106 and an inner concave surface 108. The tip portion 102 includes a curved arm 110 having one end attached to a distal end of the handle and the other end of the curved arm is the distal end of the tip portion 102b. As shown in FIG. 11, the other end of the curved arm 110 points inward toward the handle at an angle A of substantially from 40 to 60 degrees with respect to a central longitudinal reference line of the instrument. This curved arm 110 has a predetermined length 14 as measured along a straight line between a base end E5 attached to a distal end E6 of the handle H2 and the free end 102a of substantially from 14 to 15 millimeters.

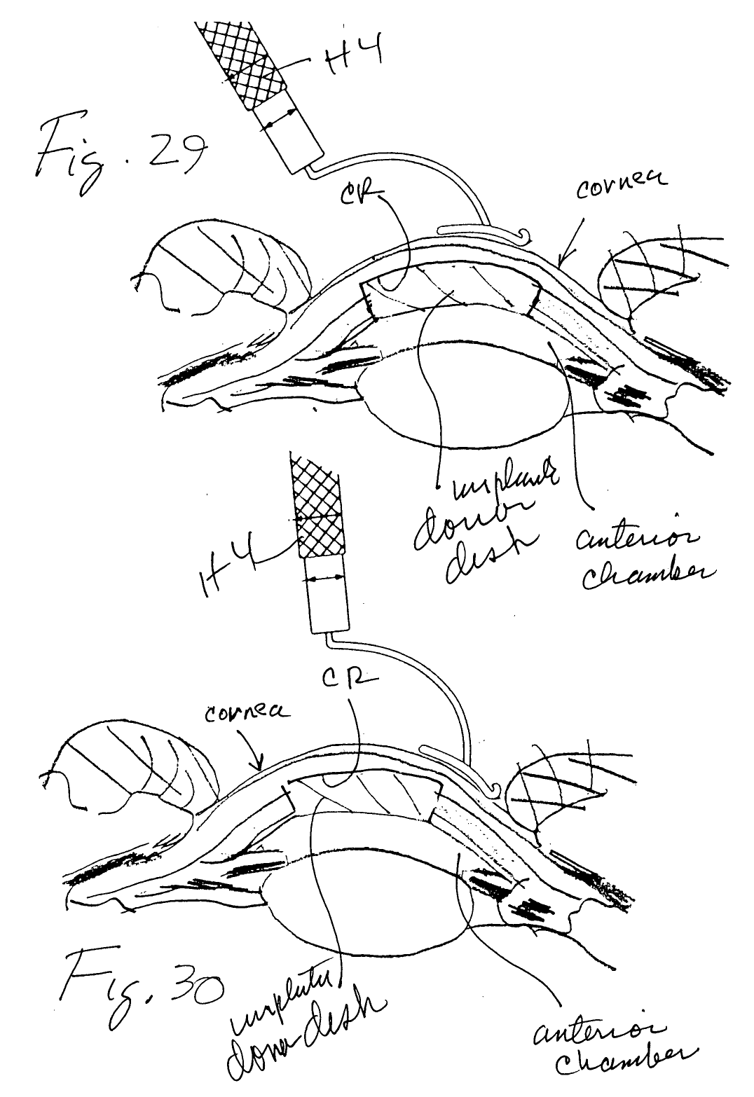

When an internal part of the precut lamellar disk PLD is adhering as shown in FIG. 27 and cannot simply be detached from the cornea's stroma by peeling away the disk from the stroma using the spatula instrument 10 as discussed above, the stripper instrument 100 is used instead after removing the spatula instrument 10 from the anterior chamber. The tip portion 102 is inserted through the incision 12 into the anterior chamber and an edge ED1 of the plate member 104 is brought to bear against the adhering internal part AP as depicted in FIG. 27. By scrapping this cutting edges against the adhering internal part AP it is detached. The tip portion is withdrawn from the anterior chamber through the incision 12 and the spatula instrument 10 is again used to wad up the remaining precut lamellar disk PLD still bonded to the stroma.

### Fixation Hook Instrument and Method of Use

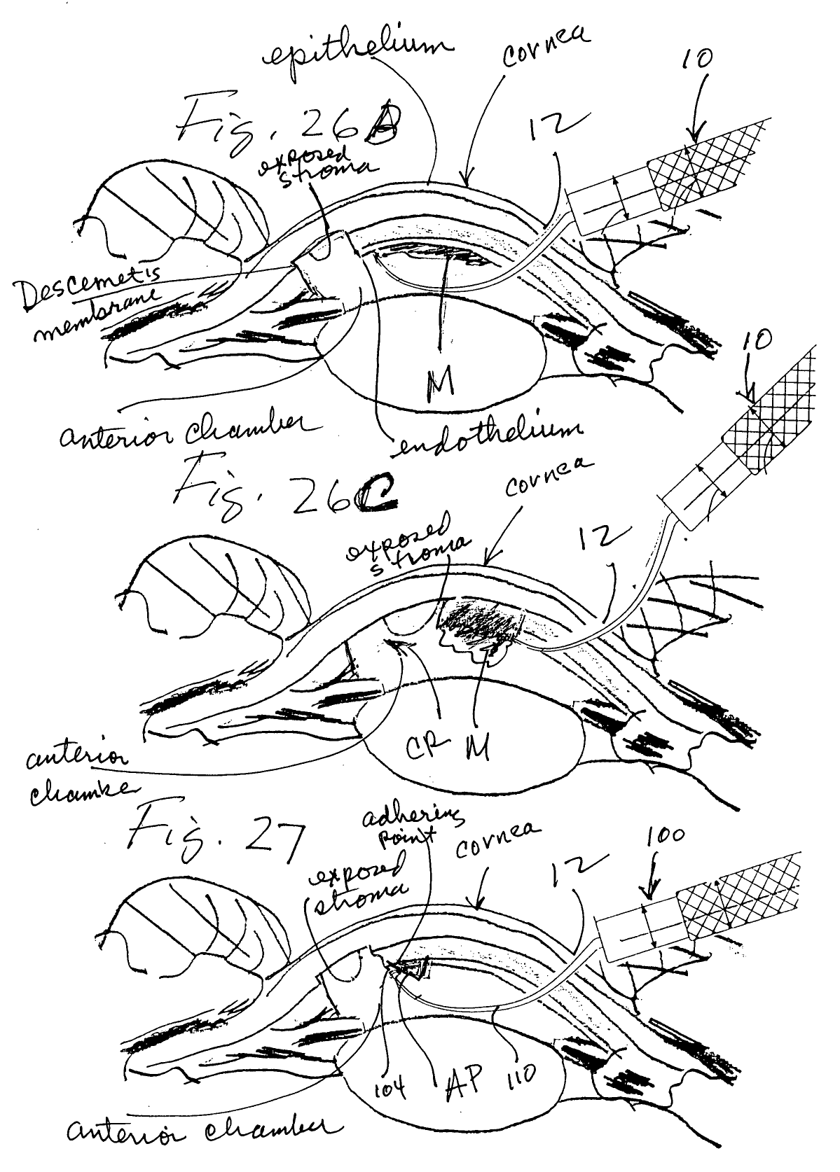

As shown in FIGS. 22 through 25, the fixation hook instrument 200 is used during unfolding of a folded donor cornea disk within the anterior chamber of the eye as depicted in FIG. 28. The fixation hook instrument 200 includes a handle H3 and a tip portion 202 comprising a curved tubular member 204 having a terminal end 204a. A flexible wire element 206 within the tubular member and mounted to be manually moveable along the tubular member 204 between a retracted position (FIG. 24) and an extended position (FIG. 25) where a terminal end of the wire element extends from the terminal end of the tubular member. The handle H3 includes a linear guideway 210 that receives a manually accessible grasping element 212 connected to an end portion of the wire element 206 extending into the handle. The grasping element 212 is manually moveable linearly along the guideway 210 to move the flexible wire element 206 within the tubular member between a retracted position and an extended position, with the wire element flexing and bending as it moves between these positions. The tip portion has a predetermined length substantially from 2.5 to 3.5 millimeters.

### Glider Instrument

As depicted in FIGS. 16 through 21 the glider instrument 300 is used to remove wrinkles in the cornea after implantation of a cornea donor disk DD as shown in FIGS. 29 and 30. This instrument 300 includes a handle H4 and a tip portion 302 comprising a plate member 306 having an external concaved surface 308 that generally conforms to the external surface of the cornea and a curved arm 310 having one end attached in a fixed central position of an underside of the plate member and another end attached to a distal end E10 of the handle. The plate member 306 has a substantially circular configuration with an edge segment folded inward to provide a substantially straight edge. The plate member 306 has a diameter substantially from 5 to 6.5 millimeters.

Any wrinkles appearing in the cornea after implantation of a cornea donor disk are ironed out by moving across and pressing against the surface of the cornea's epithelium the plate member 306. The external concaved surface 308 generally conforms to the external surface of the cornea and bears directly against said surface of the cornea's epithelium. The anterior chamber is pressurized slightly during this ironing out of wrinkles.

### SCOPE OF THE INVENTION

The above presents a description of the best mode contemplated of my surgical instruments and methods, and of the manner and process of making my instruments and using them and practicing my methods, in such full, clear, concise, and exact terms as to enable any person skilled in the art to which my instruments and methods pertain to make and use my instruments and methods. My instruments and methods are, however, susceptible to modifications and alternate constructions from the illustrative embodiments discussed above which are fully equivalent. Consequently, it is not the intention to limit my instruments and methods to the particular embodiments disclosed. On the contrary, my intention is to cover all modifications and alternate constructions coming within the spirit and scope of my as generally expressed by the following claims, which particularly point out and distinctly claim the subject matter of my instruments and methods:

1. An ophthalmic surgical instrument including
an elongated handle and a curved tip portion at an end of the handle,
said tip portion having
a predetermined configuration enabling the tip portion to be inserted into an anterior chamber of an eye through an incision along a perimeter of the eye's cornea, and
a distal free end that is pointed so, when within the anterior chamber, a surgeon can make a 360 degree substantially circular cut through the cornea's endothelium and Descemet's membrane to form a disk, and
grasp an edge of the disk and peel the disk away from the underlying stroma of the cornea to expose said stroma.

2. The surgical instrument of claim 1 where the tip portion comprises a rigid arm that
(a) tapers inwardly from a base end attached to the end of the handle to the free end,
(b) has a length as measured along a straight line between the base and free ends of substantially from 14 to 15 millimeters,
(c) a radius of curvature of substantially from 20 to 25 millimeters,
(d) at or near the base end a diameter of substantially from 3 to 4 millimeters, and
(e) at or near the free end a diameter of substantially from 0.75 to 1.2 millimeters.

3. The surgical instrument of claim 1 where the free end points inward toward the handle at an angle of substantially from 40 to 60 degrees with respect to a central longitudinal reference line of the instrument.

4. The surgical instrument of claim 1 where the elongated handle and tip portion have an overall length substantially from 100 to 120 millimeters.

5. The surgical instrument of claim 1 where
said tip portion comprises a curved rigid arm terminating in the free end,
said free end being displaced with respect to a central longitudinal reference line of the instrument and point inward toward the handle at an angle of substantially from 40 to 60 degrees with respect to a central longitudinal reference line of the instrument.

6. An ophthalmic surgical instrument including
a handle that is grasped during surgical removal of an endothelium and Descemet's membrane, and
a curved arm at a distal end of the handle, said curved arm having one end attached to the distal end of the handle and another end that
(a) is free and is configured to enter an anterior chamber of an eye, passing through an incision along the perimeter of the cornea, and
(b) is pointed to cut into the cornea's posterior, aspheric underside at a depth substantially from 10 to 20 microns when making a cut therein to provide a precut lamellar disk,
said curved arm having a predetermined length substantially from 14 to 15 millimeters, so without withdrawing the curved arm from the incision the lamellar disk, said lamellar disk being peeled away by grasping an edge thereof along the score line with the free end and removing the peeled away lamellar disk through the incision.

7. An ophthalmic surgical instrument for detaching an adhering internal part of a precut lamellar disk that cannot simply be detached from the cornea's stroma by peeling away the disk from the stroma,
said instrument including
a handle and
a curved tip portion having a predetermined configuration enabling the tip portion to be inserted into an anterior chamber of an eye through an incision along the perimeter of the cornea,
said tip portion terminating in a distal free end having a substantially T-shaped configuration including a plate member having a thin cutting edge for scrapping against the adhering internal part of the lamellar disk to detach said part.

8. The surgical instrument of claim 7 where the plate member has an overall substantially rectangular shape including an outer convex surface and an inner concave surface.

9. The surgical instrument of claim 7 where the tip portion includes a curved arm having one end attached to a distal end of the handle and the other end of the curved arm is the distal end of the tip portion.

10. The surgical instrument of claim 9 where the other end of the curved arm points inward toward the handle at an angle of substantially from 40 to 50 degrees with respect to a central longitudinal reference line of the instrument.

11. The surgical instrument of claim 9 where the curved arm has a predetermined length as measured along a straight line between a base end attached to a distal end of the handle and the free end of substantially from 14 to 15 millimeters.

12. An ophthalmic surgical instrument used during unfolding of a folded donor cornea disk within the anterior chamber of the eye, said donor cornea disk being folded so an overlapping segment covers only a part of an underlying segment to expose a surface of the underlying segment,
said instrument including
a handle and
a tip portion comprising a curved tubular member having a terminal end,
a flexible wire element within the tubular member and mounted to be manually moveable along the tubular member between a retracted position and an extended position where a terminal end of the wire element extends from the terminal end of the tubular member.

13. The surgical instrument of claim 9 where the handle includes a linear guideway that receives a manually accessible grasping element connected to an end portion of the wire element extending into the handle, said grasping element being manually moveable linearly along the guideway to move the flexible wire element within the tubular member between a retracted position and an extended position, said wire element flexing and bending as it moves between said positions.

14. The surgical instrument of claim 9 where the tip portion has a predetermined length substantially from 2.5 to 3.5 millimeters.

15. An ophthalmic surgical instrument used to remove wrinkles in the cornea after implantation of a cornea donor disk,
said instrument including
a handle and
a tip portion comprising a plate member having an external concaved surface that generally conforms to the external surface of the cornea and a curved arm having one end attached in a fixed central position of an underside of the plate member and another end attached to a distal end of the handle.

16. The surgical instrument of claim 15 where the plate member has a substantially circular configuration with an edge segment folded inward to provide a substantially straight edge.

17. The surgical instrument of claim 15 where the plate member has a diameter substantially from 5 to 6.5 millimeters.

18. An ophthalmic surgical method including the steps of
(a) making an incision along the perimeter of the cornea having a length of no greater than 5 microns,
(b) inserting through the incision a curved tip portion having a predetermined configuration enabling the tip portion to be inserted into an anterior chamber of an eye through said incision, and a distal free end that is pointed,
(c) with the curved tip portion within the anterior chamber, making a 360-degree substantially circular cut through the cornea's endothelium and Descemet's membrane to form a lamellar disk, and
(d) grasping an edge of the lamellar disk and peeling the disk away from the underlying stroma of the cornea to expose said stroma.

19. In an ophthalmic surgical procedure where a folded donor cornea disk within the anterior chamber of the eye is unfolded, said donor cornea disk being folded so an overlapping segment covers only a part of an underlying segment to expose a surface of the underlying segment,
a method of holding the disk including the steps of
(a) inserting into the cornea's anterior chamber through an incision along a perimeter of the eye's cornea a tip portion of an instrument and placing the tip portion on said exposed surface of the underlying segment,
said tip portion comprising a curved tubular member having a terminal end and housing therein a flexible wire element that is manually moveable along the tubular member between a retracted position and an extended position where a terminal end of the wire element extends from the terminal end of the curved tubular member,
said wire element being in the retracted position upon inserting the tip portion into the anterior chamber,
(b) with the tip portion on said exposed surface of the underlying segment, moving the wire element into the extended position so the terminal end of the wire element presses against the exposed surface of the underlying segment,
(c) with the wire element pressing against said exposed surface, unfolding the folded donor cornea disk by injecting air between the segments of the folded donor cornea disk, and
(d) moving the wire element into the retracted position and withdrawing the tip portion from the anterior chamber through the incision.

20. In an ophthalmic surgical procedure where an internal part of a precut lamellar disk comprising portions of the cornea's endothelium and Descemet's membrane is adhering and cannot simply detached from the cornea's stroma by peeling away the disk from the stroma,
a method including the steps of
(a) making an incision along the perimeter of the cornea having a length of no greater than 5 microns,
(b) inserting through the incision a tip portion of an instrument having a predetermined configuration enabling the tip portion to be inserted into an anterior chamber of an eye through said incision,
said tip portion terminating in a distal free end having a substantially T-shaped configuration including a plate member having an overall substantially rectangular shape including an outer convex surface and an inner concave surface and edges thin cutting edges,
(c) scrapping the cutting edges of the plate member against the adhering internal part of the lamellar disk to detach said part, and
(d) withdrawing the tip portion from the anterior chamber through the incision.

21. In an ophthalmic surgical procedure where wrinkles appearing in the cornea after implantation of a cornea donor disk,
a method of removing the wrinkles comprising the step of
moving across and pressing against the surface of the cornea's epithelium a tip portion of an ophthalmic surgical instrument comprising a plate member having an external concaved surface that generally conforms to the external surface of the cornea, said external concaved surface bearing directly against said surface of the cornea's epithelium.

22. The method of claim 21 where the anterior chamber pressurized slightly.

23. The method of claim 21 where the instrument includes a handle that is held by a surgeon performing the method and a curved arm having one end attached in a fixed central position of an underside of the plate member and another end attached to a distal end of the handle.

24. The method of claim 21 where the plate member has a substantially circular configuration with an edge segment folded inward to provide a substantially straight edge.

25. The method of claim 24 where the plate member has a diameter substantially 5 to 6.5 millimeters.
